(19) United States
(12) Patent Application Publication
(10) Pub. No.: US 2026/0057875 A1
(43) Pub. Date: Feb. 26, 2026

(54) ACTIVE NOISE CANCELLATION STETHOSCOPE

(71) Applicant: Quanta Computer Inc., Taoyuan City (TW)

(72) Inventors: Yung-Ming CHUNG, Taoyuan City (TW); Jia-Li SUNG, Taoyuan City (TW)

(21) Appl. No.: 18/947,360

(22) Filed: Nov. 14, 2024

(30) Foreign Application Priority Data
Aug. 23, 2024 (TW) 113131716

(51) Int. Cl. G10K 11/178; A61B 7/04

(52) U.S. Cl. CPC G10K 11/178; A61B 7/04

(57) ABSTRACT

An active noise cancellation stethoscope including a sound receiving device, a sound processing device, and a control unit is provided. The sound receiving device includes a main microphone and a sub microphone. The main microphone is configured to receive and output a first sound signal and the sub microphone is configured to receive and output a second sound signal. The sound processing device is coupled to the sound receiving device to receive the first and second sound signals, and is configured to convert the first and second sound signals into third and fourth sound signals in digital mode respectively. The control unit is coupled to the sound processing device, and is configured to calculate the correlation between the third and fourth sound signals in order to determine whether to perform an active noise cancellation operation.

### CROSS REFERENCE TO RELATED APPLICATIONS

This application claims priority of Taiwan Patent Application No. 113131716, filed on Aug. 23, 2024, the entirety of which is incorporated by reference herein.

### BACKGROUND OF THE INVENTION

### Field of the Invention

The present invention relates in general to a stethoscope, and in particular it relates to a stethoscope that automatically determines whether to perform an active noise cancellation operation.

### Description of the Related Art

Traditional analog stethoscopes have front and back sides for receiving sounds across different frequency ranges. However, the microphones in these stethoscopes do not only capture the desired sounds (e.g., sounds emitted by the human body), but they also pick up ambient sounds, such as the potential sirens of ambulances near a hospital, surrounding voices, and other noise. When the volume of these ambient sounds is too loud, they can interfere with the judgment about the desired sounds, or even drown them out. Therefore, there is a need for a new device to address the above issues.

### BRIEF SUMMARY OF THE INVENTION

According to an embodiment of the present disclosure, an active noise cancellation stethoscope is provided, which comprises a sound receiving device, a sound processing device, and a control unit. The sound receiving device has a main microphone and a sub microphone, wherein the main microphone is configured to receive and output a first audio signal, and the sub microphone is configured to receive and output a second audio signal. The sound processing device is coupled to the sound receiving device and configured to receive the first audio signal and the second audio signal, thereby converting the first audio signal and the second audio signal into a third audio signal and a fourth audio signal in digital mode, respectively. The control unit is coupled to the sound processing device and configured to calculate the correlation between the third audio signal and the fourth audio signal thereby determining whether to perform an active noise cancellation operation.

According to another embodiment of the present disclosure, the control unit further comprises an active noise canceller configured to perform an active noise cancellation operation, and the active noise cancellation operation comprises: simultaneously capturing a first window of the third audio signal and a second window of the fourth audio signal, respectively; calculating the correlation coefficient of the third audio signal and the fourth audio signal during the first window and second window; and comparing the correlation coefficient with a reference correlation coefficient. If the correlation coefficient is greater than or equal to the reference correlation coefficient, the control unit automatically activates the active noise canceller to perform the active noise cancellation operation, outputting a noise-canceling signal from the active noise canceller. Conversely, if the correlation coefficient is less than the reference correlation coefficient, the control unit automatically deactivates the active noise canceller to stop the active noise cancellation operation and receives the third audio signal from the audio processing device.

According to yet another embodiment of the present disclosure, the control unit further comprising an equalizer configured to receive a selection signal. When the active noise canceller is activated, the equalizer receives the noise-canceling signal and, based on the selection signal, selects the low-frequency mode, the high-frequency mode, or the mixed mode to optimize the noise-canceling signal and output a first equalized signal. Conversely, when the active noise canceller is deactivated, the equalizer receives the third audio signal and selects, based on the selection signal, the low-frequency mode, the high-frequency mode, or the mixed mode to optimize the third audio signal and output a second equalized signal.

### BRIEF DESCRIPTION OF THE DRAWINGS

The present invention can be more fully understood by reading the subsequent detailed description and examples with references made to the accompanying drawings, wherein:

FIG. 1 is a block diagram of an active noise cancellation stethoscope according to an embodiment of the present disclosure;

FIG. 2 is a schematic diagram illustrating the determination of whether to perform an active noise cancellation operation according to an embodiment of the present disclosure;

FIG. 3 is a schematic diagram illustrating the active noise cancellation operation of the active noise cancellation stethoscope according to an embodiment of the present disclosure; and

FIG. 4 is a schematic diagram illustrating the scenario in which the active noise cancellation stethoscope does not perform an active noise cancellation operation according to an embodiment of the present disclosure.

### DETAILED DESCRIPTION OF THE INVENTION

Herein, reference is made to the accompanying illustrations to describe multiple embodiments, in which similar reference numerals are used to denote similar or equivalent components. The illustrations are not necessarily drawn to scale and are intended solely for illustrative purposes to convey the aspects and features of the present disclosure. A significant amount of specific details, relationships, and methods are outlined to provide a complete understanding of the particular aspects and features of the present disclosure. However, those skilled in the relevant technical field will appreciate that these aspects and features can be practiced in the absence of one or more of the specific details, in different relationships, or using alternative methods. In some instances, well-known structures or operations are not represented in detail for clarity. The multiple embodiments disclosed herein are not necessarily limited to the order of operations or events depicted; some actions may occur in a different sequence and/or simultaneously with other actions or events. Furthermore, not all actions illustrated are essential to practicing the specific aspects and features of the present disclosure.

For the sake of clarity in this description, unless explicitly stated otherwise, singular terms include the plural and vice versa. The term “includes” means “includes but is not limited to.” Additionally, terms used to indicate approximations, such as “approximately,” “nearly,” “substantially,” “about,” and similar terms, may be used to represent “within,” “close to,” “almost at,” “within 3-5%,” “within acceptable manufacturing tolerances,” or any logical combinations thereof. Similarly, the terms “vertical” or “horizontal” also encompass “within 3-5% in the vertical or horizontal direction,” respectively. Furthermore, directional terms such as “top,” “bottom,” “left,” “right,” “above,” and “below” refer to equivalent directions as described in the referenced illustrations; positions should be understood relative to the object or component being referenced, based on the context, such as the typical position of an object or component during use, or according to other descriptions provided herein.

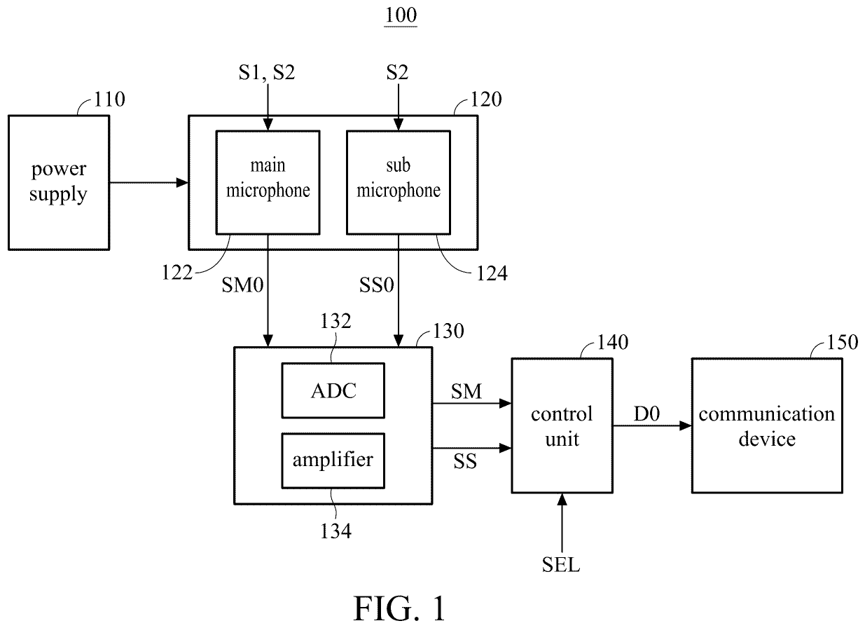

FIG. 1 is a block diagram of an active noise cancellation stethoscope 100 according to one embodiment described in the present disclosure. As shown in FIG. 1, the active noise cancellation stethoscope 100 includes a power supply 110, a sound receiving device 120, a sound processing device 130, a control unit 140, and a communication device 150.

The power supply 110 is configured to serve as the power source for the active noise cancellation stethoscope 100, such as a battery. The sound receiving device 120 includes a main microphone 122 and a sub microphone 124, wherein the main microphone 122 is configured to receive a target sound S1 (e.g., sounds emitted by the human body) and ambient sound S2, and outputs a main microphone audio signal SM0 (which contains both target sound S1 and ambient sound S2). The sub microphone is configured to receive ambient sound S2 and outputs a sub microphone audio signal SS0 (which contains only ambient sound S2). The sound processing device 130 is configured to receive the main microphone audio signal SM0 and the sub microphone audio signal SS0 from the sound receiving device 120. The sound processing device 130 amplifies these signals through an amplifier 134 (for example, amplifying the sub microphone audio signal SS0 by approximately 5 times) and converts the main microphone audio signal SM0 and the sub microphone audio signal SS0 using an analog-to-digital converter (ADC) 132. Subsequently, the sound processing device 130 output a main audio signal SM and a sub audio signal SS. Specifically, the main audio signal SM corresponds to the digital mode of the main microphone audio signal SM0, while the sub audio signal SS corresponds to the digital mode of the sub microphone audio signal SS0.

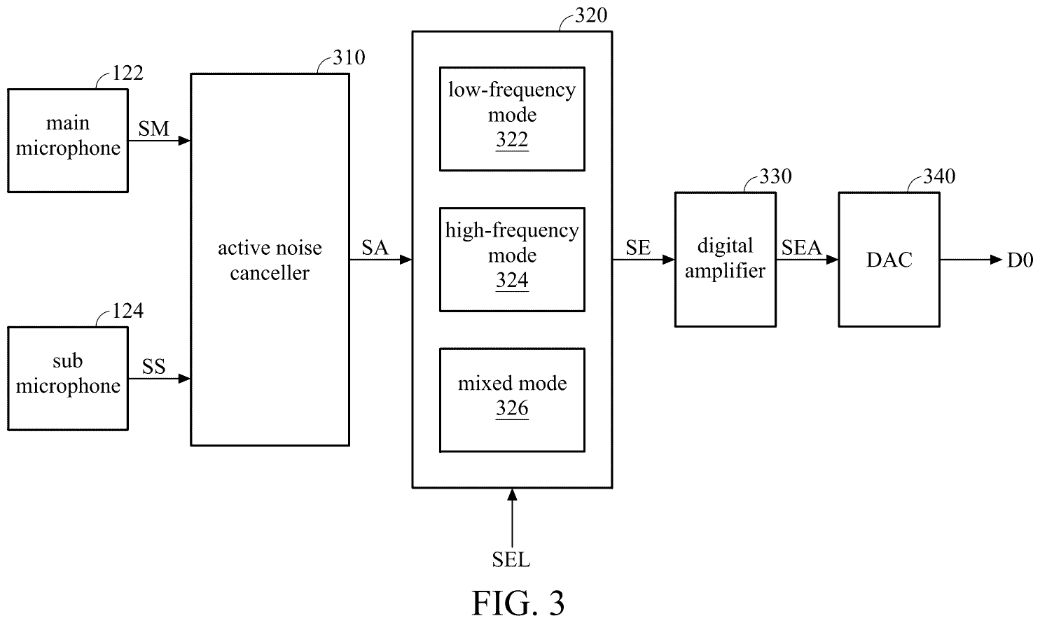

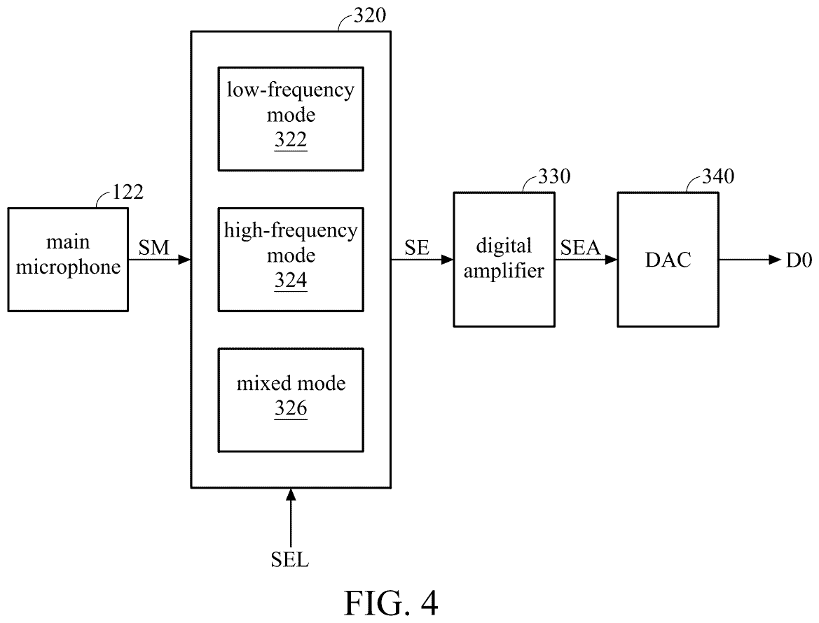

The control unit 140 is configured to perform active noise cancellation (ANC) operations on the main audio signal SM and the sub audio signal SS, as well as to filter and integrate these signals. The active noise cancellation operations will be explained in detail with reference to FIGS. 3 and 4. After the control unit 140 performs active noise cancellation and filtering on the main audio signal SM and the sub audio signal SS, it generates an output signal D0, which is then output to the communication device 150. In one embodiment, the communication device 150 may be configured to store the output signal D0 (e.g., as a memory or cloud database). In another embodiment, the communication device 150 may additionally include a display screen configured to display the output signal D0 (e.g., showing the waveform of the output signal D0), or to allow the selection of the data of the output signal D0 to be retrieved via an application within the communication device 150.

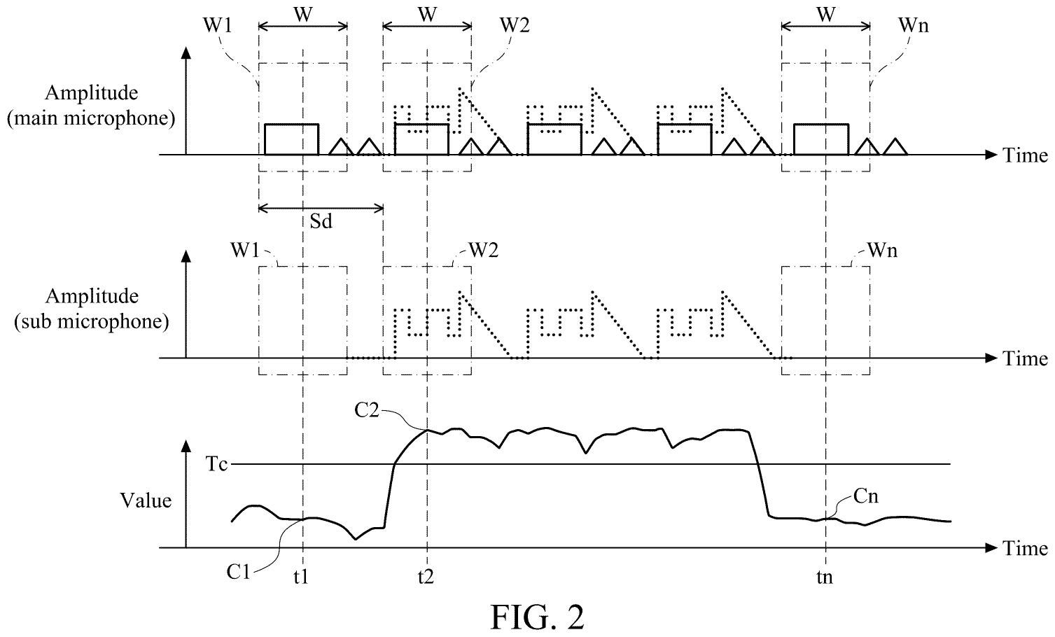

FIG. 2 is a schematic diagram illustrating the determination of active noise cancellation operations according to the embodiments described in the present disclosure. The control unit 140 can automatically start or stop the active noise cancellation operation based on the correlation between the main audio signal SM and the sub audio signal SS. As shown in FIG. 2, the topmost timing diagram displays the waveform of the main audio signal SM (with solid and dashed lines), while the middle timing diagram displays the waveform of the sub audio signal SS (shown only with dashed lines). The bottom timing diagram shows the correlation between the sounds captured by the main microphone 122 and the sub microphone 124, wherein Tc represents the reference correlation coefficient, and Cn (n=1, 2, . . . ) represents the correlation coefficient of the sounds received by the main microphone 122 and the sub microphone 124 in the n^{th} window. The correlation coefficient Cn can be calculated using the following formula:

$$Cn = \frac{{\sum }_{i = 1}^{K} ( Wsi - \overline{Ws} ) ( Wai - \overline{Wa} )}{\sqrt{{\sum }_{i = 1}^{K} {( Wsi - \overline{Ws} )}^{2} {\sum }_{i = 1}^{K} {( Wai - \overline{Wa} )}^{2}}}$$

Wherein Wsi and Wai represent the i^{th} sample points in the n^{th} window of the main microphone 122 and the sub microphone 124, respectively, and Ws and Wa represent the average values of the sample points in the nth window for the main microphone 122 and the sub microphone 124, respectively.

The method by which the control unit 140 determines whether to perform active noise cancellation operations is described as follows. First, the control unit 140 captures a first window W1 based on a predefined window time W and calculates the correlation coefficient C1 at time t1 within the first window W1 to serve as the correlation coefficient for the first window W1. Next, the correlation coefficient C1 is compared to the reference correlation coefficient Tc. As shown in FIG. 2, since the correlation coefficient C1 is less than the reference correlation coefficient Tc, the control unit 140 does not perform active noise cancellation operations during the first window W1.

In a specific embodiment, the range of the reference correlation coefficient Tc can be determined through trial and error or averaging. Testing has shown that the optimal effect is achieved when the reference correlation coefficient Tc is between 0.55 and 0.8. If the reference correlation coefficient Tc is set too low, it becomes too easy to activate ANC, resulting in distortion of the audio signal. Conversely, if the reference correlation coefficient Tc is set too high, it becomes difficult to activate ANC, leading to poor active noise cancellation performance. FIG. 2 shows the results when the reference correlation coefficient Tc is set to 0.6.

Subsequently, the control unit 140 captures a second window W2 after an interval of a window gap Sd, and calculates the correlation coefficient C2 at time t2 during a period within the second window W2 to serve as the correlation coefficient for the second window W2. Next, the correlation coefficient C2 is compared to the reference correlation coefficient Tc. As shown in FIG. 2, since the correlation coefficient C2 is greater than the reference correlation coefficient Tc, the control unit 140 will perform active noise cancellation operations during the second window W2.

Next, the control unit 140 captures the n^{th} window Wn based on the predefined window time W and calculates the correlation coefficient Cn at time tn during a period within the n^{th} window Wn to serve as the correlation coefficient for the n^{th} window Wn. Then, the correlation coefficient Cn is compared to the reference correlation coefficient Tc. As shown in FIG. 2, since the correlation coefficient Cn is less than the reference correlation coefficient Tc, the control unit 140 does not perform active noise cancellation operations during the nth window Wn.

In other words, if the correlation coefficient of the current window is less than the reference correlation coefficient Tc, the control unit 140 does not perform active noise cancellation operations during the current window. Conversely, if the correlation coefficient of the current window is greater than or equal to the reference correlation coefficient Tc, the control unit 140 performs active noise cancellation operations during the current window. Furthermore, although, as shown in FIG. 2, the first window W1 and the second window W2 do not overlap, in some embodiments, the predefined window time W and the window gap Sd can be adjusted to allow the first window W1 and the second window W2 (or any two adjacent windows) to have overlapping durations. This can reduce the sampling time difference between the correlation coefficients C1 and C2, thereby increasing the sensitivity and accuracy of the control unit 140 in determining whether to perform active noise cancellation operations.

FIG. 3 is a schematic diagram illustrating the active noise reduction operation of the active noise cancellation stethoscope 100 described in this disclosure. FIG. 3 shows the procedure for performing active noise cancellation on sound when the control unit 140 determines that the correlation coefficient Cn is greater than or equal to the reference correlation coefficient Tc. For the sake of simplicity, the power supply 110, sound processing device 130, and communication device 150 from FIG. 1 have been omitted. The main microphone 122 receives the target sound S1 and ambient sound S2, and outputs the main audio signal SM after conversion through the sound processing device 130. The sub microphone 124 receives the ambient sound S2 and outputs the sub audio signal SS after conversion through the sound processing device 130. The ambient sound S2 received by the sub microphone 124 can be amplified by the amplifier 134 in the sound processing device 130, which allows for better active noise reduction effects during subsequent active noise cancellation operations.

Next, the main audio signal SM and the sub audio signal SS are output to an active noise canceller 310 to determine whether to perform active noise cancellation operations as shown in FIG. 2. For detailed judgment procedures, please refer to FIG. 2 and the descriptions above and will not be reiterated here. After the active noise cancellation operation is completed, the active noise canceller 310 generates a noise-canceling signal SA and outputs the noise-canceling signal SA to an equalizer (EQ) 320. The equalizer 320 has three modes: a low-frequency mode 322, a high-frequency mode 324, and a mixed mode 326. The equalizer 320 can determine the current mode to be triggered based on a selection signal SEL. The selection signal SEL can be manually input by the user before starting to use the active noise cancellation stethoscope 100. Additionally, the equalizer 320 can further optimize the sound signal for different frequency ranges of the three modes using methods such as notch filters, high-pass filters (HPF), low-pass filters (LPF), or combinations of the above.

For example, as shown in FIG. 3, when the active noise canceller 310 is on (i.e., when active noise cancellation operations are needed), if the selection signal SEL indicates that the low-frequency mode 322 should be triggered, the equalizer 320 can optimize the low-frequency performance of the noise-canceling signal SA through a combination of a high-pass filter with a cutoff frequency of 200 Hz and a low-pass filter with a cutoff frequency of 1000 Hz. If the selection signal SEL indicates that the high-frequency mode 324 should be triggered, the equalizer 320 can use a combination of a high-pass filter with a cutoff frequency of 100 Hz, notch filters at 350 Hz and 1500 Hz, and a low-pass filter with a cutoff frequency of 1000 Hz, to optimize the high-frequency performance of the noise-canceling signal SA. If the selection signal SEL indicates that the mixed mode 326 should be triggered, the equalizer 320 can optimize the overall performance of the noise-canceling signal SA using a combination of notch filters with cutoff frequencies of 80 Hz and 1500 Hz, along with a low-pass filter with a cutoff frequency of 1000 Hz.

After the equalizer 320 completes the optimization of the noise-canceling signal SA, the equalizer 320 generates an equalized signal SE and outputs the equalized signal SE to a digital amplifier 330 for amplification. Then, the digital amplifier 330 produces an amplified equalized signal SEA and outputs the amplified equalized signal SEA to a digital-to-analog converter (DAC) 340. The DAC 340 converts the equalized signal SEA into an analog output signal D0 and outputs the analog output signal D0 to the communication device 150 for storage or user review. Additionally, since the digital amplifier 330 is only configured to amplify the equalized signal SE, if the amplitude of the equalized signal SE is sufficient, the use of the digital amplifier 330 can be optional.

FIG. 4 is a schematic diagram illustrating the operation of the active noise cancellation stethoscope 100 when active noise cancellation is not performed, as described in this disclosure. FIG. 4 shows the procedure for not performing active noise cancellation on sound when the control unit 140 determines that the correlation coefficient Cn is less than the reference correlation coefficient Tc. For the sake of simplicity, the power supply 110, sound processing device 130, and communication device 150 from FIG. 1 have been omitted. Additionally, since the process shown in FIG. 4 does not require active noise cancellation operations, the sub microphone 124 and the active noise canceller 310 are also omitted.

The main microphone 122 receives both the target sound S1 and the ambient sound S2, and outputs the main sound signal SM after conversion through the sound processing device 130. At this time, since the control unit 140 does not perform active noise cancellation operations, the active noise canceller 310 is turned off, and the main sound signal SM is directly output to the equalizer 320. For example, if the selection signal SEL indicates that the low-frequency mode 322 should be triggered, the equalizer 320 can use a combination of a high-pass filter with a cutoff frequency of 60 Hz, notch filters at 10 Hz and 50 Hz, and a low-pass filter with a cutoff frequency of 254 Hz, to optimize the low-frequency performance of the noise-canceling signal SA. If the selection signal SEL indicates that the high-frequency mode 324 should be triggered, the equalizer 320 can optimize the high-frequency performance of the noise-canceling signal SA using a combination of a high-pass filter with a cutoff frequency of 115 Hz and a low-pass filter with a cutoff frequency of 575 Hz. If the selection signal SEL indicates that the mixed mode 326 should be triggered, the equalizer 320 can use a combination of a high-pass filter with a cutoff frequency of 50 Hz, notch filters at 10 Hz and 60 Hz, and a low-pass filter with a cutoff frequency of 600 Hz, to optimize the overall performance of the noise-canceling signal SA.

After the equalizer 320 completes the optimization of the main sound signal SM, the equalizer 320 generates an equalized signal SE and outputs the equalized signal SE to the digital amplifier 330 for amplification. The digital amplifier 330 then produces an amplified equalized signal SEA and outputs the amplified equalized signal SEA to a digital-to-analog converter (DAC) 340. The DAC 340 converts the equalized signal SEA into an analog output signal D0 and outputs the analog output signal D0 to the communication device 150 for storage or user review. Additionally, since the digital amplifier 330 is only configured to amplify the equalized signal SE, if the amplitude of the equalized signal SE is sufficient, the use of the digital amplifier 330 can be optional.

This disclosure provides an active noise cancellation stethoscope configured to automatically determine whether to activate the active noise canceller for active noise cancellation operations based on the correlation between the sounds received by the main microphone (for capturing target sounds and ambient sounds) and the sub microphone (for capturing ambient sounds). If the correlation coefficient between the sounds received by the main and sub microphones is greater than or equal to the reference correlation coefficient, the active noise canceller is automatically activated for active noise cancellation operations. Conversely, if the correlation coefficient is less than the reference correlation coefficient, the active noise canceller is automatically turned off, and active noise cancellation operations are halted.

In addition to performing active noise cancellation operations to minimize or eliminate the impact of ambient sounds on target sounds, this disclosure further outputs the noise-reduced sound signal to an equalizer for optimization. Using a selection signal input by the user in advance, the equalizer can be set to optimize for low-frequency, high-frequency, or overall aspects of the sound signal, thereby further achieving the goal of eliminating the effects of ambient sounds. Additionally, this disclosure features the ability to automatically turn the active noise canceller on or off, ensuring that active noise cancellation operations are only conducted when needed (e.g., when ambient sounds are too loud). This not only ensures that the sound signal does not experience significant distortion due to active noise reduction but also helps to reduce power consumption in a timely manner when active noise cancellation is not required, extending the usage cycle of the active noise cancellation stethoscope.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An active noise cancellation stethoscope, comprising:
a sound receiving device, having a main microphone and a sub microphone, wherein the main microphone is configured to receive and output a first audio signal, and the sub microphone is configured to receive and output a second audio signal;
a sound processing device, coupled to the sound receiving device, and configured to receive the first audio signal and the second audio signal, and convert the first audio signal and the second audio signal into a third audio signal and a fourth audio signal in digital mode, respectively; and
a control unit, coupled to the sound processing device, and configured to calculate a correlation between the third audio signal and the fourth audio signal to determine whether to perform an active noise cancellation operation.

2. The active noise cancellation stethoscope as claimed in claim 1, wherein the first audio signal corresponds to a target sound and an ambient sound, while the second audio signal corresponds to the ambient sound.

3. The active noise cancellation stethoscope as claimed in claim 1, wherein the sound processing device comprises:
an amplifier, configured to amplify the second audio signal; and
an analog-to-digital converter, configured to convert the first audio signal and the amplified second audio signal into the third audio signal and the fourth audio signal, respectively.

4. The active noise cancellation stethoscope as claimed in claim 1, wherein the control unit further comprises an active noise canceller configured to perform the active noise cancellation operation, and the active noise cancellation operation includes:
simultaneously capturing a first window of the third audio signal and a second window of the fourth audio signal;
calculating a correlation coefficient of the third audio signal and the fourth audio signal during the first window and second window; and
comparing the correlation coefficient with a reference correlation coefficient,
wherein, if the correlation coefficient is greater than or equal to the reference correlation coefficient, the control unit automatically activates the active noise canceller to perform the active noise cancellation operation and outputs a noise-canceling signal from the active noise canceller; and
wherein, if the correlation coefficient is less than the reference correlation coefficient, the control unit automatically deactivates the active noise canceller to stop the active noise cancellation operation and receives the third audio signal from the sound processing device.

5. The active noise cancellation stethoscope as claimed in claim 4, wherein the control unit further comprises an equalizer configured to receive a selection signal, and wherein:
when the active noise canceller is activated, the equalizer receives the noise-canceling signal and, based on the selection signal, selects a low-frequency mode, a high-frequency mode, or a mixed mode to optimize the noise-canceling signal and output a first equalized signal; and
when the active noise canceller is deactivated, the equalizer receives the third audio signal and selects one of the low-frequency mode, the high-frequency mode, and the mixed mode based on the selection signal to optimize the third audio signal and outputs a second equalized signal.

6. The active noise cancellation stethoscope as claimed in claim 5, further comprising a digital amplifier coupled to the equalizer and configured to receive and amplify the first equalized signal or the second equalized signal, and output an amplified first equalized signal or an amplified second equalized signal to a digital-to-analog converter.

7. The active noise cancellation stethoscope as claimed in claim 6, wherein the digital-to-analog converter converts the amplified first equalized signal or the amplified second equalized signal to generate an output signal and outputs the output signal to a communication device.

8. The active noise cancellation stethoscope as claimed in claim 5, further comprising a digital-to-analog converter configured to convert the first equalized signal or the second equalized signal to generate an output signal and output the output signal to a communication device.

9. The active noise cancellation stethoscope as claimed in claim 7, wherein the communication device is configured to store the output signal or display the output signal through a screen.

10. The active noise cancellation stethoscope as claimed in claim 8, wherein the communication device is configured to store the output signal or display the output signal through a screen.

11. The active noise cancellation stethoscope as claimed in claim 1, further comprising a power supply, wherein the power supply is a battery.
